(19) United States
(12) Patent Application Publication
(10) Pub. No.: US 2020/0078273 A1
(43) Pub. Date: Mar. 12, 2020

(54) METHOD AND APPARATUS FOR REMOVAL OF CONTAMINANTS FROM SURFACES

(71) Applicant: Michael J. McKinnon-Dane, Dover, NH (US)

(72) Inventor: Michael J. McKinnon-Dane, Dover, NH (US)

(21) Appl. No.: 16/562,704

(22) Filed: Sep. 6, 2019

Related U.S. Application Data
(60) Provisional application No. 62/728,540, filed on Sep. 7, 2018.

(51) Int. Cl. A61K 8/04; A61Q 19/10; A61K 8/365; A61K 8/49

(52) U.S. Cl. CPC A61K 8/046; A61Q 19/10; A61K 2800/87; A61K 8/49; A61K 8/365

(57) ABSTRACT

Cationic surfactant foams, methods for producing cationic surfactant foams, and methods for removal of contaminants from surfaces such as human skin using such foams are disclosed. A cationic surfactant alone or a combined solution of a cationic surfactant and a weak acid are pumped through a mechanical pump and intermixed with air to produce the cationic surfactant foams. In one of the embodiments, the cationic surfactant is isostearamidopropyl morpholine lactate (ISML) and the weak acid is citric acid.

### TECHNICAL FIELD

This disclosure generally relates to formulations, foams, methods, and apparatuses for removing contamination, such as metal contamination, from surfaces including dermal surfaces of humans or other animals.

### BACKGROUND

Exposure to metals may occur in a wide variety of locations, including in the workplace, in homes or schools, or in the outdoor environment. Skin contact is one significant route of exposure to metals or metal compounds, because (i) metals may be absorbed through the skin; (ii) skin can act as a reservoir for metals; (iii) skin surface deposition can be an important source of secondary contamination; and (iv) impairment or loss of skin barrier function can occur. Unfortunately, many metals typically are not easily washed off the skin. Surveys of industrial wash rooms show most potentially contaminated workers invest less than 60 seconds cleaning up before breaks. However, most conventional industrial soaps require 5 to 10 minutes of continuous washing to reduce surface concentrations of powdered metals (such as lead) to levels that are not likely to present potential health hazards. Thus, significant metal contamination may remain after washing.

Most methods of removing lead or other potentially harmful metals from a solid surface involve harsh physical processes (e.g., hot air blowers combined with scraping) and/or caustic chemicals (such as, methylene chloride, toluene, acetone, calcium hydroxide, magnesium hydroxide, and sodium hydroxide; see, e.g., U.S. Pat. No. 5,964,961). These methods are unsuitable for applications where no substantial modification or damage to the surface is desired; such as, for example, removal of lead (or other metals) from human skin.

Products that claim to remove lead from human skin contain active ingredients such as EDTA or anionic surfactants. EDTA is a suspected persistent environmental pollutant and a skin irritant, which may cause reddening or inflammation on prolonged skin contact. Anionic surfactants may also cause skin irritation, such as dryness and scaling. Moreover, anionic surfactants may not be fully effective in removing lead contamination.

It is desirable, therefore, to provide safe, reliable and effective compositions and methods for removing metals, such as lead, from surfaces, including human skin. Of particular need are compositions and methods that do not substantially damage the treated surface, or unduly irritate biological surfaces, such as skin.

### SUMMARY

According to an embodiment, a method for removing metals from a surface is disclosed. The method includes foaming a cationic surfactant using a mechanical pump to produce a cationic surfactant foam and applying the cationic surfactant foam to the surface to remove a contaminant from the surface.

One or more of the following features may be included. The cationic surfactant foam may include a weak acid. The weak acid may be selected from citric acid, acetic acid, ascorbic acid, glycolic acid, maleic acid, fumaric acid, benzoic acid, salicylic acid, nicotinic acid, cinnamic acid, tartaric acid, mandelic acid, and a combination thereof. The weak acid may be citric acid. The concentration of the citric acid in the foam may be between 0.01 and 0.1 M. The ratio of the cationic surfactant and the weak acid in the foam may be in between 10:1 and 50:1. The cationic surfactant may include isostearamidopropyl morpholine lactate (ISML), lapryium chloride, cetyltrimethylammonium bromide, di-dodecyldimethylammonium bromide, trimethylbenzylammonium chloride, diethyl ester dimethyl ammonium chloride, hexadecyltrimethylammonium, tetradecyl trimethyl ammonium bromide, cetylpyridinium chloride, alkyl C₁₂-C₁₄-dimethylbenzyl ammonium chloride, dodecyl pyridinium chloride, 11-(acryloyloxy)undecyl(trimethyl)ammonium bromide, dimethyl dioctadecyl ammonium bromide, N-alkyl dimethylbenzyl ammonium chloride, or a combination thereof. The cationic surfactant may include ISML. The concentration of the ISML in the foam may be from 0.1 to 0.5 M. The foam may have a pH of greater than 2 and less than 7. The pH of the foam may be between about 5 to about 6.5. The foam may be produced without a foaming agent. The surface may be a non-porous surface. The non-porous surface may include a dermal surface. The dermal surface may be a human skin. The contaminant may include at least one of a metal, a microorganism or a particle. The metal may include at least one of lead, cadmium, tin, barium, arsenic, chromium, copper, mercury, silver, zinc, strontium, thallium, germanium, zirconium, or combinations thereof. The metal may include lead. The lead may be metallic lead, lead ions, lead alloys, lead-containing compounds, and combinations thereof. The cationic surfactant foam may remove at least 95% of at least one metal from the surface in a single washing. The foam may be applied directly to the surface. The foam may be applied without a wipe or a cloth. The method may further include rinsing the surfactant from the surface with water.

According to another embodiment, a method for producing a foam is disclosed. The method includes providing a cationic surfactant in a pump, wherein the cationic surfactant is in liquid form and pumping the cationic surfactant and air through a mechanical pump to produce a foam.

One or more of the following features may be included. The method may further include providing a weak acid. The cationic surfactant may include ISML. The weak acid may be citric acid.

According to yet another embodiment, a foam consisting of a cationic surfactant, water and a week acid is disclosed.

One or more of the following features may be included. The cationic surfactant may include ISML. The week acid may be citric acid.

### BRIEF DESCRIPTION OF THE DRAWINGS

FIG. 1 is a cross-sectional view of an example foam dispenser incorporating a mechanical pump of the present disclosure.

The FIGURE depicts one of the example embodiments of the present disclosure for purposes of illustration only. Numerous variations, configurations, and other embodiments will be apparent from the following detailed discussion. Like reference symbols in the various drawings indicate like elements.

### DETAILED DESCRIPTION

### Overview

The present disclosure relates to methods of removal of a metal or metals from surfaces, in particular, from the dermal surfaces. The methods described herein involve application of a cationic foam to surfaces exposed to metal contamination. The foam may be produced by pumping a cationic surfactant through a mechanical pump.

Foam is a material formed by trapping pockets of gas in a liquid or solid. In most foams, the volume of gas is larger than that of the liquid or solid, with thin films of the liquid or solid separating the regions of gas. In foams, liquids or solids divide gas into gas bubbles/pockets of different sizes to produce polydisperse foams. In some embodiments, the foam density may be in the range of about 0.01 g/cm³ to about 0.25 g/cm³. The stability of a foam is critical for its various applications. The stability of a foam is defined by its half-decay time, which is the time taken by a foam to reach half of the initial height of the foam. In some embodiments, the stability of the foam may be greater than about 10 min, about 60 min, about 120 min or about 400 min.

The foam disclosed herein can be produced from, at least in part, cationic surfactants. In some embodiments, the cationic surfactant may be a liquid or may be dissolved in a suitable solvent, such as water. In one embodiment, the cationic surfactant may include a single cationic surfactant while in other embodiments, the cationic surfactant may include two or more cationic surfactants.

In another embodiment, the foam disclosed herein can be produced from, at least in part, a non-ionic surfactant. In one instance, the foam may include a single non-ionic surfactant while in other instances, the non-ionic surfactant may include two or more non-ionic surfactants.

In yet another embodiment, the foam may include a cationic surfactant in combination with one or more non-ionic surfactants. In some other embodiments, the foam may include a cationic or a non-ionic surfactant in combination with some other ingredients.

A “surfactant” (also called a “surface acting agent) is a substance that lowers the surface or interfacial tension of the medium in which the surfactant is dissolved. Surfactant molecules have a hydrophilic portion, which associates with water, and a hydrophobic portion, which avoids water. In the absence of other hydrophobic molecules, the hydrophobic portion of the surfactant molecule protrudes from the surface of a water drop. As a result, the water molecules in the drop are disrupted and surface tension decreases so that the water drop no longer beads up, but spreads. The hydrophobic end of a surfactant molecule, which protrudes from the water drop, is also free to attach to hydrophobic molecules or particles (such as, grease, fat, or oil) on the surface.

A cationic surfactant can be defined as a surfactant molecule that can dissociate to yield a surfactant ion whose polar group is positively charged. Cationic surfactants include, for example, quaternary ammonium derivatives (e.g., aliphatic, aromatic and heterocyclic quaternaries and their respective salts such as benzalkonium chloride or alkylaryl quaternary salt); amidoamines (e.g., amidoamine salts and oxides); and betaines (e.g., N-alkylbetaines).

A non-ionic surfactant consists of a hydrophilic head and a hydrophobic tail and carry no charge. Non-ionic surfactant does not dissociate when dissolved in water, and has the broadest range of properties depending upon the ratio of hydrophilic-lipophilic balance. Non-ionic surfactant includes, for example, fatty alcohol ethoxylates (e.g., octaethylene glycol monododecyl ether); alkyl phenol ethoxylates (e.g., Triton X-100 and nonoxynols); fatty acid ethoxylates; ethoxylated amines and or fatty acid amides; terminally blocked ethoxylates; fatty acid esters of glycerol; fatty acid esters of sorbitol (e.g., Tween, sorbitan monostearate, and sorbitan monolaurate; alkyl polyglucosides; amine oxides; sulfoxides; and phosphine oxides.

In differing embodiments, the cationic surfactants include one or more of isostearamidopropyl morpholine lactate (ISML), lapryium chloride, cetyltrimethylammonium bromide, di-dodecyldimethylammonium bromide, trimethylbenzylammonium chloride, diethyl ester dimethyl ammonium chloride, hexadecyltrimethylammonium, tetradecyl trimethyl ammonium bromide, cetylpyridinium chloride, alkyl C₁₂-C₁₄-dimethylbenzyl ammonium chloride, dodecyl pyridinium chloride, 11-(acryloyloxy)-undecyl-(trimethyl)-ammonium bromide, dimethyl dioctadecyl ammonium bromide, N-alkyl dimethylbenzyl ammonium chloride, or combinations thereof. In one embodiment, the cationic surfactant is isostearamidopropyl morpholine lactate (ISML), which can dissociate to yield a positively charged isostearamidopropyl morpholine group and a functional lactic acid ion group.

In another embodiment, the cationic surfactant may also be mixed with a weak acid to form an acidic combined. In some other embodiments, the cationic surfactant and the weak acid may be in the form of separate solutions. A cationic surfactant foam of a lower pH has been shown to improve the removal of heavy metals such as lead from surfaces such as human skin.

The weak acid is an acid that, upon transient exposure, does not (i) cause undue skin irritation in a living organism, or (ii) substantially damage the surface of a tangible object. Transient exposure to a weak acid may be as brief as about one minute, about two minutes or up to about five minutes; however, depending on the nature of the particular weak acid, exposures of up to about 60 minutes, or even up to about 24 hours or longer may be sufficiently transient to avoid undue skin irritation or substantial damage to another surface by the weak acid. Undue skin irritation may include, for example, cracking of the skin surface, bleeding, ulcers, or prolonged redness, itching, soreness, or dryness of the skin. Substantial damage to the surface of a tangible object may include, for example, etching or pitting of the surface, or removal of or damage to any finish in or on the surface (such as, paint, varnish, shellac, or dye).

The weak acids may include, for example, citric acid, acetic acid, ascorbic acid, glycolic acid, maleic acid, fumaric acid, benzoic acid, salicylic acid, nicotinic acid, cinnamic acid, tartaric acid, mandelic acid, or combinations thereof. In one embodiment, the weak acid is citric acid.

In certain embodiments, the pH of the combined solution of cationic surfactant and weak acid (such as ISML and citric acid) or the separate solution of weak acid (such as citric acid) is greater than 1.5 or greater than 2.0 and less than about pH 6.5, less than about pH 5.0, less than about pH 4.5, less than about pH 4.0, or less than about pH 3.5. In this one embodiment, the pH of the combined solution of cationic surfactant and weak acid (such as ISML and citric acid) or the separate solution of weak acid (such as citric acid) may be in the range of about pH 3.0 to about pH 6.5, or about pH 3.0 to about pH 4.0.

Different ratios of a cationic surfactant to a weak acid can be used. In some embodiments, however, a weight ratio between the total amount of cationic surfactant and the total amount of weak acid can be greater than 2, greater than 5, greater than 10, greater than 20, greater than 25, greater than 50, or greater than 100. In one embodiment, weight ratio between cationic surfactant (such as, ISML) and weak acid (such as, citric acid) is about 25.

In some embodiments, the concentration of cationic surfactant can be greater than 0.05M, greater than 0.1M, greater than 0.15M, greater 0.2M, greater than 0.25M, greater than 0.5M, or greater than 1.0M. In one specific embodiment, concentration of the cationic surfactant (such as, ISML) can be about 0.25M. In some other embodiments, concentration of the weak acid can be greater than 0.01M, greater than 0.02M, greater than 0.03M, greater than 0.04M, greater than 0.05M, greater than 0.075M, or greater than 0.1M. In one embodiment, the concentration of the weak acid can be 0.05M.

The solution(s) may also include a variety of other components that may assist in providing the desired cleaning properties. For example, additional components may include water, other surfactants, emollients, preservatives, chelating agents, pH buffers, fragrances, colorants, or combinations thereof.

In the present disclosure, cationic surfactant foams, instead of wipes or cloths, are used for removal of metal contamination. Generally, cationic surfactants don't foam well as cationic surfactants are not compatible with conventional foaming agents. Cationic surfactants are also incompatible with anionic surfactants. As all soaps are anionic based, cationic surfactants cannot be used effectively with soaps. However, the inventor has surprisingly found that a mechanical pump can be used to produce foam/lather without the use of a foaming agent. The foam/lather can be used directly out of a bottle to remove contaminants such as metals or metal ions from surfaces such as human hands. Foam can electrostatically displace metal or metal ions resulting in a better and more hygienic user experience.

As mentioned earlier, the foam may be produced by pumping a cationic surfactant and a gas such as air through a mechanical pump. A mechanical pump may be defined as a device that moves liquids or gases or slurries by mechanical action. Pumps may be classified into three categories based on the methods they use to move the fluid: direct lift, displacement, and gravity pumps.

Mechanical pumps consume energy to perform mechanical work of moving the liquid or gas. Pumps operate via many energy sources, including manual operation and electricity. In one embodiment, the mechanical pump is operated by manual operation.

Mechanical pumps may be submerged in the liquid or gas they are pumping or they may be placed external to the liquid or gas. In one embodiment, the mechanical pump is in at least partial contact with the cationic surfactant solution or with the combined solution of the cationic surfactant and the weak acid.

When a manually operated mechanical hand pump is activated, the cationic surfactant solution or the combined solution is pushed through a small nozzle which entrains air into the solution and foams the surfactant. In another embodiment, the mechanical pump may act as a dual pump to push both the cationic solution and air through small opening to create a lather. Foam or lather can be produced through the pumping mechanism of the mechanical pump in absence of any foaming agent.

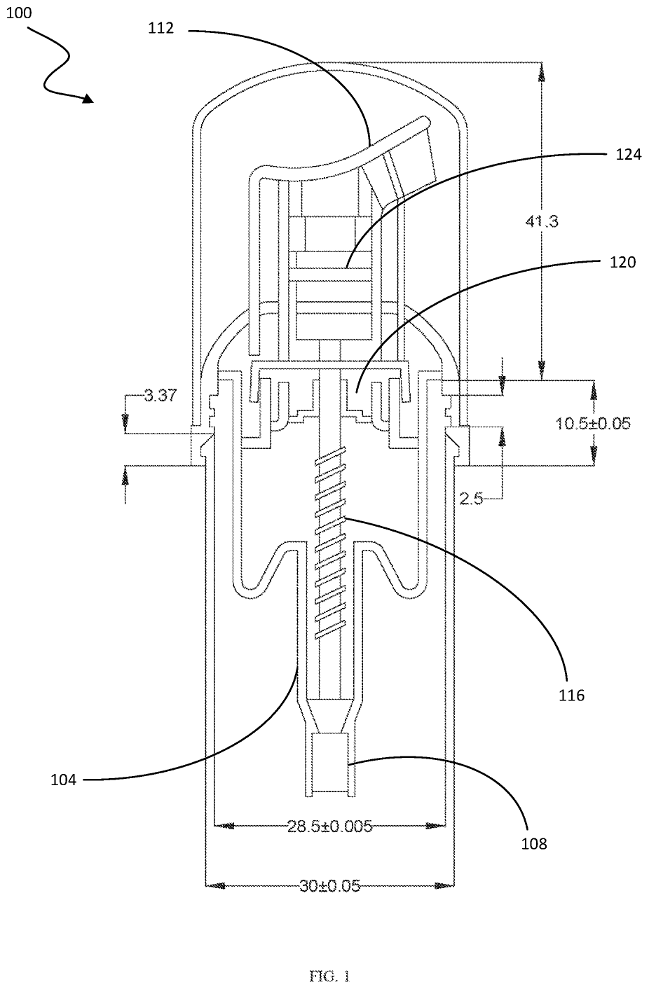

FIG. 1 is a cross-sectional view of an example foam dispenser 100 incorporating a mechanical pump 104. The foam dispenser 100 includes mechanical pump 104 which is attached to a tube 108 that is placed in a surfactant solution (not shown). When spout 112 is pushed down, the surfactant contained in the chamber including spring 116 get pushed up. However, when the spout 112 is released, the surfactant is drawn up through the tube 108 and into a pump chamber 120. When the spout 112 is depressed it also pumps air into the pump chamber 120. In the pump chamber 120, liquid constituents (including surfactants and water) and air are mixed and subsequently discharged through a mesh 124 (usually a nylon mesh).

The neck size of the foam dispensing mechanical pump is bigger than the neck size of other types of non-foaming pumps, to accommodate the pump chamber. The usual neck size of a pump chamber ranges from about 40 mm to about 43 mm. In one embodiment, the neck size is 41.3 mm

The foam can remove contaminants from variety of surfaces. A surface can be defined as a non-porous and/or non-dispersible outer layer of an organism or a tangible, solid object. Representative surfaces may include dermal surfaces (such as hands), floors, walls, windowsills, clothing, laundry, shoes, equipment surfaces, and furniture surfaces. A “dermal surface” refers to the skin (such as the strateum corneum) of an animal, including, for example, mammals (such as humans). More generally, “integument” refers to the outer protective surface covering an animal or a plant.

The contaminants that can be removed from surfaces include potentially harmful metals, microorganism (e.g., bacteria and viruses), and particles among other contaminants.

The potentially harmful metals include metals, metal ions, or metal-containing compound (or other chemical form of a metal) with the potential to adversely affect the health or wellbeing of living organisms (such as humans), which are exposed to the metal. The harmful effect of the metal on the living organism may be observed relatively soon after exposure to the metal, or may occur over an extended period of time. The adverse effects of a potentially harmful metal may result after a single exposure or after multiple exposures. Potentially harmful metals often, but not always, have a relatively high atomic mass; for example, lead is a relatively high mass metal that has a known potential to harm living organisms, while beryllium is an example of a relatively low mass potentially harmful metal.

Representative potentially harmful metals include lead, cadmium, tin, barium, beryllium, arsenic, chromium, copper, lead, mercury, silver, zinc, strontium, thallium, cobalt, germanium, or zirconium. In some embodiments, a metal cation having a +2 charge (such as, Pb²⁺, Ba²⁺, Be²⁺, Co²⁺, Ni²⁺, Pd²⁺, Pt²⁺, Hg²⁺, Zn²⁺, Cd²⁺, or Sn²⁺) is a potentially harmful metal. In other examples, metal oxides, such as AsO₃, MnO, MnO₂, PbO₂, Pb₃O₄, or particular lead-containing compounds, such as PbCl₂ or PbCO₃, are potentially harmful metals.

Lead includes elemental lead, lead in any ionic or nonionic state and lead in any compound form (such as lead oxides or lead halides). Particular examples of lead include Pb⁰, Pb²⁺, PbO₂, Pb₃O₄, PbCl₂ or PbCO₃.

Removal of a metal from a surface can include displacing an amount or concentration of metal from the surface (such as, a dermal surface) by physical or chemical methods or a combination thereof.

The foams work as chemical mechanisms to remove metal contamination, for instance, from skin or other surfaces. In some examples, chemical mechanisms of removal can include solubilization, ionization, alteration of pH and/or chelation of a metal contaminant.

In some embodiments, the foam will contain sufficient amounts or concentrations of cationic surfactant and/or weak acid to solubilize a metal. Solubilize is defined herein to mean to dissolve or to disperse on the molecular level into a solvent or suspension.

In some embodiments, the amounts or concentrations of cationic surfactant and/or weak acid may be sufficient to solubilize at least one of lead, cadmium, tin, barium, arsenic, chromium, copper, lead, mercury, silver, zinc, strontium, thallium, cobalt, germanium, zirconium, ionic forms of any thereof (such as, +2 cations), compounds containing any thereof (such as, oxides or halides thereof), or combinations thereof.

In some embodiments, the amount of cationic surfactant(s) and weak acid(s) applied to a surface (in separate solutions or a combined solution) in the form of a foam is from about 0.0001 ml/cm² to about 0.05 ml/cm²; for instance, from about 0.0005 ml/cm² to about 0.025 ml/cm², from about 0.001 ml/cm² to about 0.01 ml/cm², or from about 0.002 ml/cm² to about 0.005 ml/cm². In one embodiment, about 0.0036 ml/cm² of a combined solution including cationic surfactant (such as, ISML) and weak acid (such as citric acid) is applied to a surface (such as, human hands).

In some other embodiments, a metal is solubilized when at least about 50%, at least about 60%, at least about 75%, at least about 80%, at least about 90%, at least about 95%, at least about 98%, or even 100% of the metal on the treated surface is dissolved by a cationic surfactant, weak acid or a combination of both present in the combined foam solution, and/or by the combination of cationic surfactant, weak acid and mechanical displacement resulting from rinsing the foamed surface with water. In another instance, the foam solubilizing metal or metal ions or other contaminants at the treated surfaces can be simply wiped out with a paper towel or a fabric towel. In other instances, a metal is solubilized when the amount or concentration of metal remaining on the surface is less than a permissible exposure limit (PEL) for the metal of interest; for example, at least about 5%, at least about 10%, at least about 15%, at least about 25%, at least about 50%, at least about 75%, at least about 80%, or at least about 90% less than the PEL. The permissible exposure limit (PEL) is a legal limit in the United States for exposure of an employee to a chemical substance or physical agent such as loud noise.

### Methods for Removing Metals

Methods for removing metals from a surface are disclosed herein. Such methods include applying a foam of a cationic surfactant or a foam of a combined solution of a cationic surfactant and a weak acid to a surface, and rinsing the surface with water. In some cases, the foam can be directly applied to the surface without the use of any wipe or cloth, and metals can be effectively removed from the skin using only the foam and water.

In some embodiments, foam of the cationic surfactant(s) and/or weak acid(s) can be applied one or more times directly to a surface; then the surface is rinsed with water. In some other embodiments, foam of the cationic surfactant(s) and/or weak acid(s) can be applied directly to a surface once, and then the surface is rinsed with water. In another embodiment, the steps of direct application of foam of the cationic surfactant(s) and/or weak acid(s) onto the surface and rinsing the surface with water, can be repeated few times.

Applying foam and rinsing steps (collectively, washing step) of a disclosed method may be as brief or prolonged as is needed to remove a metal from a contaminated surface. For example, the duration of a washing step to get below the PEL may require no more than a few seconds (such as, no more than about 10 seconds, no more than about 15 seconds, no more than about 30 seconds or no more than 60 seconds). In other cases, the surfaces may be washed for a minute or more.

In one embodiment, a user dispenses foamed cationic surfactant directly from a pump into the palm of the user's hand. In a manner similar to that used when washing with liquid soap, the user can cover both hands with the cationic surfactant by rubbing hands together after dispensing about 10 to 50 mL of foam onto one hand. The cationic surfactant solubilizes any heavy metals that are on the user's hands. Once bound to the surfactant, the heavy metals can be rinsed away from the skin using tap water. The user's hands are typically not dried out as when applying an anionic surfactant, yet the cationic surfactant is sufficiently soluble in water that it can be easily rinsed away with water. The process can be repeated one or more times to reduce the heavy metal concentration even more. The foam can help to spread the surfactant over the entire hand and allows for removal between fingers and other areas that might be neglected when a wipe is used.

It is contemplated that the disclosed methods may be useful to remove any metal(s) that may be solubilized in the presence of a foam of a cationic surfactant and/or weak acid. In some method embodiments, lead, cadmium, tin, barium, arsenic, chromium, copper, lead, mercury, silver, zinc, strontium, thallium, cobalt, germanium, zirconium, ionic forms of any thereof (such as, +2 cations), compounds containing any thereof (such as, oxides or halides thereof), or combinations thereof may be removed. Lead, lead ions, or lead-containing compounds (such as lead oxides or lead halides) or combinations thereof can be removed in some embodiments.

### Further Consideration

The foregoing description of the embodiments of the disclosure has been presented for the purpose of illustration; it is not intended to be exhaustive or to limit the claims to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments is intended to be illustrative, but not limiting, of the scope of the invention, which is set forth in the following claims.

Unless otherwise explained, all technical and scientific terms used herein have the same meaning as commonly understood by one of ordinary skill in the art to which the disclosed subject matter belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means including A or B, or including A and B. It is further to be understood that any quantitative values are approximate whether the word “about” or “approximately” or the like are stated or not. All publications, patent applications, patents, and other references mentioned herein are incorporated by reference in their entirety. In addition, the materials, methods, and examples described herein are illustrative only and not intended to be limiting.

What is claimed is:

1. A method for removing metals from a surface, the method comprising:
foaming a cationic surfactant using a mechanical pump to produce a cationic surfactant foam; and
applying the cationic surfactant foam to the surface to remove a contaminant from the surface.

2. The method of claim 1 wherein the cationic surfactant foam comprises a weak acid.

3. The method of claim 2, wherein the weak acid is selected from citric acid, acetic acid, ascorbic acid, glycolic acid, maleic acid, fumaric acid, benzoic acid, salicylic acid, nicotinic acid, cinnamic acid, tartaric acid, mandelic acid, and a combination thereof.

4. The method of claim 3, wherein the weak acid is citric acid.

5. The method of claim 4, wherein concentration of the citric acid in the foam is between 0.01 and 0.1 M.

6. The method of claim 2, wherein the ratio of the cationic surfactant and the weak acid in the foam is in between 10:1 and 50:1

7. The method of claim 1, wherein the cationic surfactant comprises isostearamidopropyl morpholine lactate (ISML), lapryium chloride, cetyltrimethylammonium bromide, di-dodecyldimethylammonium bromide, trimethylbenzylammonium chloride, diethyl ester dimethyl ammonium chloride, hexadecyltrimethylammonium, tetradecyl trimethyl ammonium bromide, cetylpyridinium chloride, alkyl C₁₂-C₁₄-dimethylbenzyl ammonium chloride, dodecyl pyridinium chloride, 11-(acryloyloxy)undecyl(trimethyl)ammonium bromide, dimethyl dioctadecyl ammonium bromide, N-alkyl dimethylbenzyl ammonium chloride, or a combination thereof.

8. The method of claim 7, wherein the cationic surfactant comprises ISML.

9. The method of claim 8, wherein concentration of the ISML in the foam is from 0.1 to 0.5 M.

10. The method of claim 1, wherein foam has a pH of greater than 2 and less than 7.

11. The method of claim 10, wherein the pH of the foam is between about 5 to about 6.5.

12. The method of claim 1, wherein the foam is produced without a foaming agent.

13. The method of claim 1, wherein the surface is a non-porous surface.

14. The method of claim 13, wherein the non-porous surface comprises a dermal surface.

15. The method of claim 14, wherein the dermal surface is human skin.

16. The method of claim 1, wherein the contaminant comprises at least one of a metal, a microorganism or a particle.

17. The method of claim 16, wherein the metal comprises at least one of lead, cadmium, tin, barium, arsenic, chromium, copper, mercury, silver, zinc, strontium, thallium, germanium, zirconium, or combinations thereof.

18. The method of claim 16, wherein the metal comprises lead.

19. The method of claim 18, wherein the lead is metallic lead, lead ions, lead alloys, lead-containing compounds, and combinations thereof.

20. The method of claim 1, wherein the cationic surfactant foam removes at least 95% of at least one metal from the surface in a single washing.
